(19) United States
(12) Patent Application Publication
(10) Pub. No.: US 2013/0289056 A1
(43) Pub. Date: Oct. 31, 2013

(54) METHODS OF ADMINISTERING AN EGFR INHIBITOR

(71) Applicants: Robert Michael Lorence, Bethesda, MD (US); Mehdi Shahidi, Sutton (GB); Peter Stopfer, Warthausen (DE)

(72) Inventors: Robert Michael Lorence, Bethesda, MD (US); Mehdi Shahidi, Sutton (GB); Peter Stopfer, Warthausen (DE)

(73) Assignee: Boehringer Ingelheim International GmbH, Ingelheim am Rhein (DE)

(21) Appl. No.: 13/817,571

(22) PCT Filed: Aug. 24, 2011
(86) PCT No.: PCT/US11/48922

§ 371 Date: Apr. 23, 2013

Related U.S. Application Data
(60) Provisional application No. 61/377,177, filed on Aug. 26, 2010.

(51) Int. Cl. A61K 31/517; C12Q 1/68; A61K 31/426; G01N 33/74

(52) U.S. Cl. CPC A61K 31/517; G01N 33/74; C12Q 1/6886; A61K 31/426
USPC 514/266.24; 435/7.23; 435/6.11

(57) ABSTRACT

Disclosed are methods of treating a patient having cancer with an EGFR inhibitor requiring an active step of avoiding or modifying the co-administration of such drugs with P-gp modulators. This invention also relates to a medicament for treating a patient having cancer, comprising an EGFR inhibitor as the active product ingredient, customized for avoiding co-administration with P-gp inducers and/or inhibitors by an instruction added to the medicament or the package containing said medicament.

### APPLICATION DATA

This application claims benefit to U.S. provisional application Ser. No. 61/377,177 filed Aug. 26, 2010.

### BACKGROUND OF THE INVENTION

1. Technical Field

This invention relates to a method of administering an EGFR inhibitor(s) to a patient having cancer in which there is an active step of avoiding or modifying co-administration of the EGFR inhibitors with P-glycoprotein (P-gp) modulators, i. e. with P-gp inducers or P-gp inhibitors.

2. Background Information

Epidermal growth factor receptor (EGFR) tyrosine kinase inhibitors have been studied clinically to demonstrate efficacy in treating certain cancers. Compounds which inhibit signal transduction by tyrosine kinases, for example of the human EGF receptor, have been shown to be useful for treating pathophysiological processes caused by hyperfunction of tyrosine kinases. David W. Fry, Pharmacol. Ther. Vol. 82, Nos. 2-3, pp. 207-218, 1999. Several irreversible inhibitors have been shown to have therapeutic advantages such as prolonged tumor suppression compared to reversible inhibitors such as gefitinib. DeBono & Rowinsky, Br. Med. Bull. 64:227-254 (2002). Compounds have been disclosed in WO 02/50043, WO 2004/074263 and WO 2005/037824 as dual inhibitors of erbb1 receptor (EGFR) and erbB2 (Her2/neu) receptor tyrosine kinases, suitable for the treatment of e.g. benign or malignant tumours, particularly tumours of epithelial and neuroepithelial origin, metastasis and the abnormal proliferation of vascular endothelial cells (neoangiogenesis), for treating diseases of the airways and lungs which are accompanied by increased or altered production of mucus caused by stimulation by tyrosine kinases, as well as for treating diseases of the gastrointestinal tract and bile duct and gall bladder which are associated with disrupted activity of the tyrosine kinases. BIBW 2992 is a highly selective, potent, irreversible tyrosine inhibitor of EGFR and HER2 with promising effects having been seen in non-small cell lung cancer (NSCLC) patients. Drugs of the Future 2008, 33(8): 649-654; Li, D. et al, Oncogene (2008) 27, 4702-4711. BIBW 2992 pharmacokinetic characteristics (PK) after single and multiple doses revealed moderately fast absorption British Journal of Cancer (2008) 98,80-85. BIBW 2992 exhibited more than dose-proportional increase in exposure in healthy volunteers and in cancer patients.

Skin toxicity and diarrhea were the most common adverse events in the preliminary results of a Phase II clinical trial in patients with adenocarcinoma of the lung and activating EGFR mutations. Mukherji, D., et al, Expert Opin. Investig. Drugs (2009) 18(3), 293-300.

P-gp is a transmembrane efflux pump protein and appears to be an important component of the barrier which protects tissues from potentially harmful substances by pumping them out of cells. Standard chemotherapy treatment regimens for cancer typically involve highly toxic compounds designed to kill tumor cells. In multi-drug resistance (MDR), tumor cells will use P-gp to pump out therapeutic medication before it can penetrate the tumor and effectively eliminate the cancer. Juranka P F et al., FASEB J. 1989 December; 3(14):2583-92. While this has led to the development of agents that inhibit the action of P-gp, this approach has been unpredictable since many randomized controlled trials evaluating P-gp substrate drugs used in combination with or without P-gp modulators, have not shown significant improvements in outcome. Ferry D R et al., Eur J Cancer. 1996 June; 32A(6): 1070-81. Therefore there may be other factors limiting the effectiveness of P-gp inhibitors in vivo. One study has shown that P-gp inhibitors effectiveness varied according to cell distances from blood vessels: P-gp overexpressing tumors had increased uptake of doxorubicin in proximal cells, minimal or no effect on drug uptake at intermediate distances from blood vessels but decreased drug uptake in more distal cells. Patel, Krupa J. et al., BMC Cancer 2009; 9: 356. Work done until now on P-gp modulators being used to increase effectiveness of chemotherapeutic drugs has shown limitations and unpredictability of such of P-gp modulators and suggest the importance of considering drug distribution in the design and development of novel treatment strategies.

### BRIEF SUMMARY OF THE INVENTION

The work cited above supports the principle that treating a patient having cancer with an EGFR inhibitor requires an active step of (1) avoiding or modifying the co-administration of such drugs with P-gp modulators; or (2) modifying the dose of EGFR inhibitor.

### DETAILED DESCRIPTION OF THE INVENTION

The present inventors have discovered for the first time that taking both drugs together, a P-gp inhibitor and an EGFR inhibitor results in an increased drug exposure of said EGFR inhibitor. A direct corollary to this discovery is that taking both drugs together, a P-gp inducer and EGFR inhibitor results in a decreased drug exposure of the EGFR inhibitor. In either case, the simultaneous administration of a P-gp inhibitor/inducer together with a EGFR inhibitor should be avoided and/or modified or the dose of the EGFR inhibitor should be modified.

Alternatively, administration of a potent P-gp inhibitor at least 6 hours after an EGRF inhibitor administration may minimize the DDI (drug drug interaction) which was seen during concomitant intake of EGFR inhibitor together with a P-gp inhibitor/inducer. The invention therefore provides

a method of treatment with an EGRF inhibitor, the method comprising
(a) identifying a patient in need of treatment with an EGRF inhibitor, preferably BIBW 2992;
(b) determining that the patient is receiving therapy with a P-gp modulator;
(c) causing the patient's therapy with the P-gp modulator to cease prior to treatment with the EGRF inhibitor;
(d) administering the EGRF inhibitor to the patient;
(e) resuming therapy with a P-gp modulator not earlier than 6 hours after the EGRF inhibitor administration.

Another embodiment of the invention therefore provides a method of treatment with an EGRF inhibitor, the method comprising
(a) identifying a patient in need of treatment with an EGRF inhibitor, preferably BIBW 2992;
(b) determining that the patient is receiving therapy with a P-gp modulator;
(c) causing the patient's therapy with the P-gp modulator to cease for the duration of treatment with the EGRF inhibitor; and
(d) administering the EGRF inhibitor to the patient.

A further embodiment of the invention provides a method of treatment with an EGRF inhibitor, the method comprising
(a) identifying a patient in need of treatment with an EGRF inhibitor; preferably BIBW 2992
(b) determining that the patient is receiving therapy with a P-gp modulator;
(c) modifyingmodifying, preferably reducing the administering of said P-gp modulator before starting of administering the EGFR inhibitor; and
(d) administering the EGRF inhibitor to the patient.

A further embodiment of the invention provides a pharmaceutical composition comprising an EGRF modulator, preferably BIBW 2992, for use in treating a cancer patient by a method comprising
(a) determining that the patient is receiving therapy with a P-gp modulator;
(b) causing the patient's therapy with the P-gp modulator to cease for the duration of treatment with the EGRF inhibitor; and
(d) administering the EGRF inhibitor to the patient.

A further embodiment of the invention provides a pharmaceutical composition comprising an EGRF modulator, preferably BIBW 2992, for use in treating a cancer patient by a method comprising
(a) determining that the patient is receiving therapy with a P-gp modulator;
(b) modifying, preferably reducing the administering of said P-gp modulator before starting of administering the EGFR inhibitor; and
(c) administering the EGRF inhibitor to the patient.

The P-gp modulator is either a potent P-gp inhibitor or a potent P-gp inducer and the EGFR inhibitor is a reversible or an irreversible EGFR inhibitor. Preferably, the P-gp modulator is a potent P-gp inhibitor and the EGFR inhibitor is an irreversible EGFR inhibitor, more preferably said EGFR inhibitor is BIBW 2992.

The invention therefore provides a method of treatment with BIBW 2992, the method comprising
(a) administering BIBW 2992 to a patient who is receiving therapy with a P-gp inhibitor;
(b) detecting symptoms of BIBW 2992 adverse events in the patient; and
(c) modifyingmodifying, preferably reducing or ceasing therapy with the P-gp inhibitor, while continuing therapy with BIBW 2992.

A further embodiment of the invention provides a pharmaceutical composition comprising BIBW 2992 for use in treating a cancer patient by a method comprising
(a) administering BIBW 2992 to a patient who is receiving therapy with a P-gp inhibitor;
(b) detecting symptoms of BIBW 2992 adverse events in the patient; and
(c) modifying, preferably reducing or ceasing therapy with the P-gp inhibitor, while continuing therapy with BIBW 2992.

In a preferred embodiment, this invention relates to a method of treating a patient having cancer with BIBW 2992 in which there is an active step of avoiding, if possible, co-administration of BIBW 2992 with P-gp modulators which consist of inducers and inhibitors.

In a preferred embodiment, this invention relates to a method of treating a patient having cancer with an EGFR inhibitor, in which there is an active step of stopping, if possible, administration of P-gp inducers or inhibitors prior to administration of said EGFR inhibitor, and wherein said EGFR inhibitor is preferably an irreversible EGFR inhibitor and more preferably said EGFR inhibitor is BIBW 2992.

In another embodiment, this invention relates to a method of treating a patient having cancer with an EGFR inhibitor, preferably BIBW 2992 in which the dose of the EGFR inhibitor is adjusted if there is co-administration of the EGFR inhibitor with P-gp inducers or inhibitors.

In a further preferred embodiment, this invention relates to a medicament for treating a patient having cancer, comprising an EGFR inhibitor, preferably BIBW2992, as the active product ingredient, customized for avoiding co-administration with P-gp inducers or inhibitors by an instruction added to the medicament.

The invention therefore provides a method of treatment with BIBW 2992, the method comprising
(a) identifying a patient in need of treatment with BIBW 2992;
(b) providing the patient and/or the patient's caregiver with a list of drugs that are P-gp modulators;
(c) advising the patient and/or the patient's caregiver not to treat the patient with any of the listed drugs for the duration of treatment with BIBW 2992, preferably the P-gp modulator and BIBW 2992 should not be administered at the same time; and
(d) administering BIBW 2992 to the patient.

A further embodiment of the invention provides a pharmaceutical composition comprising BIBW 2992 for use in treating a cancer patient by a method comprising
(a) providing the patient and/or the patient's caregiver with a list of drugs that are P-gp modulators;
(b) advising the patient and/or the patient's caregiver not to treat the patient with any of the listed drugs for the duration of treatment with BIBW 2992 preferably the P-gp modulator and BIBW 2992 should not be administered at the same time; and
(c) administering BIBW 2992 to the patient.

In one aspect of the invention, there is provided a method of using an EGFR inhibitor, preferably BIBW 2992 for the treatment of cancer, preferably as either a first line or second line or third line treatment of said cancer in a human patient so as to reduce either the occurrence of EGFR inhibitor toxicity/adverse events or progression of said cancer when said patient is receiving administration of P-gp modulators prior to administration of an EGFR inhibitor, said method comprising:

administering an EGFR inhibitor once daily dosage amount to the patient for treatment of cancer where there is a concomitant P-gp modulator administration followed by monitoring

i) the progression of the cancer in the patient at a time point after the patient has initiated an EGFR inhibitor treatment, wherein progression of the cancer is indicative of cancer that is not responsive to the treatment regimen; and

ii) monitoring the adverse events of an EGFR inhibitor treatment in the patient at a time point after the patient has initiated EGFR inhibitor treatment by measuring the amount and severity of adverse events as determined by, for example, Cancer Therapy Evaluation Program, Common Terminology Criteria for Adverse Events (CTCAE) version 3.0, DCTD, NCI, NIH, DHHS, Mar. 31, 2003, published Aug. 9, 2006 (http://ctep.cancer.gov/protocoldevelopment/electronic_applications/docs/ctcaev3.pdf);

wherein a progression of the cancer in i) requires either a) actively avoiding administration of P-gp modulators and/or b) increasing the EGFR inhibitor daily dosage amount;

or wherein an unacceptable level of adverse events of an EGFR inhibitor treatment in ii) requires either a) actively avoiding modifying, preferably reducing administration of P-gp modulators and/or b) reducing the EGFR inhibitor daily dosage amount.

In one aspect of the invention, there is provided a method of using an EGFR inhibitor for the treatment of cancer, preferably as a first line, second line or third line treatment of said cancer in a human patient so as to reduce the occurrence of EGFR inhibitor adverse events when said patient is receiving administration of P-gp modulator prior to administration of an EGFR inhibitor, said method comprising:

administering an EGFR inhibitor once daily dosage amount to the patient for treatment of cancer where there is a concomitant P-gp modulator administration followed by decreasing the EGFR inhibitor dose if the P-gp modulator is an inhibitor, or increase the EGFR inhibitor dose if the P-gp modulator is an inducer.

In another aspect of the invention, there is provided a pharmaceutical composition comprising an EGFR inhibitor for use in the treatment of cancer, preferably as a first line or second line or third line treatment of said cancer in a human patient so as to reduce the occurrence of either EGFR inhibitor adverse events or the progression of the cancer when said patient is receiving administration of P-gp modulators prior to administration of said EGFR inhibitor, by a method comprising:

administering an EGFR inhibitor daily dosage amount to the patient for treatment of cancer where there is a concomitant P-gp modulator administration followed by monitoring

i) the progression of the cancer in the patient at a time point after the patient has initiated EGFR inhibitor treatment, wherein progression of the cancer is indicative of cancer that is not responsive to the treatment regimen; and

ii) monitoring the adverse events of EGFR inhibitor treatment in the patient at a time point after the patient has initiated EGFR inhibitor treatment by measuring the amount and severity of adverse events as determined by, for example, Common Terminology Criteria for Adverse Events (CTCAE) version 3 grading published Aug. 9, 2006 (http://ctep.cancer.gov/protocoldevelopment/electronic_applications/docs/ctcaev3.pdf);

wherein a progression of the cancer in i) requires either a) actively avoiding administration of P-gp modulators and/or b) increasing the EGFR inhibitor daily dosage amount;

or wherein an unacceptable level of adverse events of EGFR inhibitor treatment in ii) requires either a) actively avoiding/reducing administration of P-gp modulators and/or b) reducing the EGFR inhibitor daily dosage amount.

In another aspect of the invention, there is provided a method of using an EGFR inhibitor for the treatment of cancer in a human patient so as to reduce the occurrence of EGFR inhibitor adverse events when said patient is receiving, prior to administration of said EGFR inhibitor, administration of a P-gp modulator said method comprising:

administering an EGFR inhibitor and actively avoiding by the patient P-gp modulator administration.

In another aspect of the invention there is provided

a method of optimizing therapeutic efficacy of an EGFR inhibitor for treatment of cancer in a human patient, comprising:

(a) determining that the patient is being administered an inhibitor or inducer of P-glycoprotein (P-gp);

(b) modifying, preferably reducing or avoiding completely the administering of said P-gp inhibitor or inducer before starting of administering an EGFR inhibitor; and

(c) administering the EGFR inhibitor to a subject having said cancer.

In another aspect of the invention there is provided

a method of reducing adverse events associated with treatment of cancer, comprising:

(a) determining that the patient is being administered an inhibitor of P-glycoprotein (P-gp);

(b) reducing or avoiding completely the administering of said P-gp modulator or before starting of administering an EGFR inhibitor; and

(c) administering the EGFR inhibitor to a subject having said cancer.

In a further embodiment, this invention relates to a medicament for treating a patient having cancer, comprising an EGFR inhibitor as the active product ingredient, customized for avoiding co-administration with P-gp inducers and/or inhibitors by an instruction added to the medicament or the package containing said medicament.

In yet another aspect of the invention, there is provided a pharmaceutical composition comprising an EGFR inhibitor for the treatment of cancer in a human patient so as to reduce the occurrence of either EGFR inhibitor adverse events or progression of said cancer when said patient is receiving, prior to administration of said EGFR inhibitor, administration of P-gp modulators, by a method comprising:

administering a EGFR inhibitor and actively avoiding by the patient P-gp modulator administration.

In another aspect of the invention there is provided

a pharmaceutical composition for optimizing therapeutic efficacy of an EGFR inhibitor for treatment of cancer in a human patient, by a method comprising:

(a) determining that the patient is being administered an inhibitor or inducer of P-glycoprotein (P-gp);

(b) modifying, preferably reducing or avoiding completely the administering of said P-gp inhibitor or inducer before starting of administering an EGFR inhibitor; and

(c) administering the EGFR inhibitor to a subject having said cancer.

In another aspect of the invention there is provided

a pharmaceutical composition for reducing either adverse events associated with treatment of cancer or progression of cancer, by a method comprising:

(a) determining that the patient is being administered an inhibitor or inducer of P-glycoprotein (P-gp);

(b) modifying, preferably reducing or avoiding completely the administering of said P-gp inhibitor or inducer before starting of administering an EGFR inhibitor; and

(c) administering the EGFR inhibitor to a subject having said cancer.

In another aspect of the invention there is provided an EGFR inhibitor for use in the treatment of cancer wherein said use excludes the concomitant use of a P-gp modulator.

In another embodiment there is provided the EGFR inhibitor as described above, wherein

(a) the patients are EGFR inhibitor naïve cancer patients,

(b) the patients have a tumor expressing the wild-type EGFR,

(c) the patients have a tumor expressing mutated forms of the EGFR,

(d) the patients have previously been treated with an EGFR inhibitor, and the improvement includes to overcome primary or acquired resistance to EGFR inhibitors, preferably:

(e) the patients have acquired resistance to treatment with EGFR inhibitors, wherein the improvement includes to overcome said resistance, or

(g) the patients have primary or acquired resistance caused by T790M (T790M+), wherein the improvement includes to prevent or overcome resistance to EGFR inhibitor treatment, and/or

(h) the patients have primary or acquired resistance not caused by T790M (T790M−), wherein the improvement includes to to prevent/overcome resistance to EGFR inhibitor treatment.

In another embodiment there is provided the EGFR-inhibitor according to any of the embodiments above, wherein the concomitant use of said P-gp modulator which is excluded encompasses simultaneous use, a prior use of said P-gp modulator and a later use of said P-gp modulator.

In another embodiment there is provided the EGFR inhibitor according to any of the embodiments above, wherein said EGFR inhibitor is BIBW 2992 and said cancer is NSCLC.

In another embodiment there is provided the EGFR inhibitor according to any any of the embodiments above, wherein said EGFR inhibitor is to be administered with a dosage of about 10 to 50 mg/day, preferably about 20 to 50 mg/day, more preferably 40 mg/day, preferably in the form of a tablet, taken once daily.

In another embodiment there is provided the EGFR inhibitor according to any any of the embodiments above, wherein the EGFR inhibitor is to be administered without food which shall be understood to mean at least one hour before a meal until at least 3 hours after a meal.

In another embodiment there is provided the EGFR inhibitor according to any any of the embodiments above, wherein the EGFR inhibitor is formulated as a dispersible tablet/granules/pellets/powder.

In another embodiment there is provided the EGFR inhibitor according to any of the embodiments above, wherein the dispersible tablet/granules/pellets/powder is dispersible in an aqueous solvent, preferably water.

In another embodiment there is provided the EGFR inhibitor according to any of the embodiments above, wherein the EGFR inhibitor is formulated for administration orally after dispersing or disolving said inhibitor, preferably with stirring, for at least 5, preferably at least 10, even more preferred at least 5 min in an aqueous solvent, preferably water.

In another embodiment there is provided the EGFR inhibitor according to any of the embodiments above, wherein the EGFR inhibitor is formulated for administration through a naso-gastric tube.

In another embodiment there is provided a pharmaceutical kit, comprising the EGFR inhibitor according to any of the embodiments above, customized for avoiding concomitant administration with P-gp modulators by an instruction added to the kit.

In another aspect of the invention there is provided an EGFR inhibitor for use in the treatment of cancer with a dosage regime which is adjusted according to adverse events of said EGFR inhibitor treatment, concomitant with P-gp modulator treatment, wherein said adverse events is monitored at a time point after the patient has initiated EGFR inhibitor treatment by measuring the amount and severity of adverse events as determined by, for example, with CTCAE version 3.0 grading (http://ctep.cancer.gov/protocoldevelopment/electronic_applications/docs/ctcaev3.pdf).

In another embodiment there is provided an EGFR inhibitor according to the above embodiment, wherein a progression of the cancer in i) requires either a) actively avoiding administration of P-gp modulators and/or b) increasing the EGFR inhibitor daily dosage amount or wherein an unacceptable level of adverse events of an EGFR inhibitor treatment requires either a) actively avoiding/modifying administration of P-gp modulators and/or b) reducing the EGFR inhibitor daily dosage amount.

In another aspect of the invention there is provided an epidermal growth factor receptor (EGFR) inhibitor according to any of the embodiments above, for use in the treatment of cancer wherein in said treatment EGFR inhibitor is the sole active anti-cancer ingredient.

In another aspect of the invention there is provided an in vitro method for diagnosis of a patient who is eligible for treatment with an EGFR inhibitor, wherein the method comprises the following steps:

determining in a sample obtained from the patient whether the patient has a tumor expressing wild type or mutated forms of EGFR, and

determining from data collected regarding said patient whether the patient is not under a P-gp modulator medication;

and wherein the patient is eligible for EGFR inhibitor treatment only if both above determinations can be answered affirmative.

In another embodiment there is provided an in vitro method for improving outcome of an EGFR inhibitor therapy and/or improving patient compliance during an EGFR inhibitor therapy according to the above embodiment, wherein
a determination step is carried out which encompasses providing data on a patient as to whether said patient is under a P-gp modulator therapy, and
in case the patient is under P-gp modulator therapy, providing instructions to the patient to discontinue said medication or to adjust, preferably reduce said medication or to increase the medication with an EGFR inhibitor.

In another aspect of the invention there is provided a method for monitoring cancer during an EGFR inhibitor treatment, wherein said method comprises the following steps:

providing a sample obtained from the patient to monitoring the progression or regression of disease during the EGFR inhibitor treatment, and

discontinuing a P-gp modulator treatment or adjust, preferably reducing the daily dosage thereof or increasing the daily dosage of the EGFR inhibitor.

### Definitions

All terms as used herein in this specification, unless otherwise stated, shall be understood in their ordinary meaning as known in the art. Other more specific definitions are as follows:

The terms “ErbB1”, “epidermal growth factor receptor” and “EGFR” are used interchangeably herein and refer to native sequence EGFR as disclosed, for example, in Carpenter et al. Ann. Rev. Biochem. 56:881-914 (1987), including variants thereof (e.g. a deletion mutant EGFR as in Humphrey et al. PNAS (USA) 87:4207-4211 (1990)). erbB 1 refers to the gene encoding the EGFR protein product. As used herein, the EGFR protein is disclosed as GenBank accession no. NP.sub.—005219 which is encoded by the erbB1 gene, GenBank accession no. NM.sub.—005228. See also W. J. Gullick et al., 1986, Cancer Res., 46:285-292;S. Cohen et al., 1980, J. Biol. Chem., 255:4834-4842; A. B. Schreiber et al., 1983, J. Biol. Chem., 258:846-853).

EGFR inhibitor includes reversible or irreversible EGFR inhibitors.

“Reversible EGFR” inhibitors include: structural classes 4-anilinoquinazolines, 4-[aralkylamino]pyridopyrimidines, and 4-phenylaminopyrrolo-pyrimidines. See David W. Fry, Pharmacol. Ther. Vol. 82, Nos. 2-3, pp. 209-211,1999. Specific examples include Gefitinib (compound ZD1839 “IRESSA”), Erlotinib (compound OSI-774, “TARCEVA”), Lapatinib, conventional cancer treatment with both IRESSA and TARCEVA involves the daily, oral administration of no more than 500 mg of the respective compounds.

“Irreversible EGFR inhibitor” includes any compound which binds irreversibly to EGFR, preferably to cysteine 773 of EGFR. Nonlimiting examples include compounds disclosed in U.S. Pat. No. 6,002,008, U.S. Pat. No. 7,019,012, U.S. Pat. No. 6,251,912, WO 02/50043, WO 2004/074263, WO 2005/037824, BIBW2992, EKB-569, HKI-272, HKI-357, C1-1033, Icotinib or PF-00299804, including pharmaceutical acceptable salts thereof.

Preferably BIBW 2992 dimaleate: 2-Butenamide, N-[4-[(3-chloro-4-fluorophenyl)amino]-7-[[(3S)-tetrahydro-3-furanyl]oxy]-6-quinazolinyl]-4-(dimethylamino)-, (2E)-, (2Z)-2-butendioate (1:2)

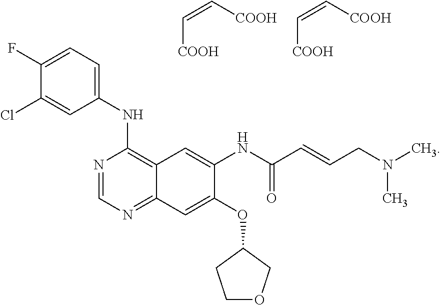 [SMILES: CN(C)C/C=C/C(=O)Nc1cc2c(Nc3ccc(F)c(Cl)c3)ncnc2cc1O[C@H]1CCOC1 ; O=C(O)/C=C\C(=O)O ; O=C(O)/C=C\C(=O)O]

P-gp, is P-glycoprotein is encoded by the ABCB1 gene (Ueda K, Clark D P, Chen C J, Roninson I B, Gottesman M M, Pastan I (January 1987). “The human multidrug resistance (mdr1) gene. cDNA cloning and transcription initiation”. J. Biol. Chem. 262 (2): 505-8. PMID 3027054).

P-gp modulators include modulators or inducers as defined herein, preferably such compounds that are potent modulators or inducers of P-gp.

Potent P-gp modulators include any agent capable of preferably potently inhibiting P-gp. Nonlimiting examples include alfentanil, amiloride, amiodarone, amitripyline, astemizole, atovaquone, atorvastatin, azelastine, azidopine, azithromycin, bepidil, biricodar, bromocriptine, carbamazepine, carvedilol, chloroquine, chlorpromazine, clarithromycin, cyclosporin, cyproheptadine, darunavir, desethylamiodarone, desipramine, dexniguldipine, dexrazoxane, diltiazem, dipyridamole, disulfiram, doxazosin, elicridqr, emetine, erythromycin, felodipine, fenofibrate, fentanyl, flavonoids, fluoxetine, fluphenazine, fluvoxamine, fucidin, gallpamil, glyburide, gramicidin D, grapefruit juice, garlic, green tea, haloperidol, hydrocortisone, hyroxyzine, josamycin, ketoconazole, imipramine, itraconazole, ivermectin, ketoconazole, laniquidar, lansoprazole, levothyroxin, lidocaine, loperamide, lopinavir, loratadine, lovastatin, maprotiline, mefloquine, methadone, mibefradil, midazolam, mitomycin C, nefazodone, nelfinavir, nicardipine, nitrendipine, nobilitin, norverapamil, omeprazole, orange juice, ofloxacin, paroxetine, pantoprazole, phenothiazines, phenobarbital, piperine, pimozide, probenecid, progesterone, promethazine, propafenone, propranolol, quercetin, quinacrine, quinidine, quinine, reserpine, ritonavir, saquinavir, sertraline, simvastatin, spironolactone, sufentanil, tacrolimus, tamoxifen, tariquidar, telithromycin, terfenadine, testosterone, tetrabenzine, thioridazine, trifluoperazine, trifluopromazine, trimipramine, valinomycin, vanadate, venlafaxine, verapamil, vinblastine, FK506, RU486 (mifepristone), valspodar PSC 833, zosuquidar and 2n-propylquinoline and combinations thereof. Preferably Cyclosporin, Erythromycin, Ketoconazole, Itraconazole, Quinidine, Phenobarbital salt with Quinidine, Ritonavir, Valspodar or Verapamil.

P-gp inducers include any agent capable of preferably potently inducing P-gp. Nonlimiting examples include Hypericum perforatum, amitriptyline, amprenavir, ASA, bromocriptine, chlorambucil, cisplatin, clotrimazole, colchicine, cyclosporine, delavirdine, daunorubicin, dexamethasone, doxorubicin, efavirenz, erythromycin, etoposide, flurouracil, hydroxyurea, insulin, lopinavir, methotrexate, mitoxantrone, morphine, nefazodone, nelfinavir, nevirapine, nicardipine, nifedipine, paclitaxel, phenobarbital, phenothiazines, phenytoin, prazosin, probenecid, reserpine, retinoid acid, ritonavir, rifampin, rifampicin, St. John's Wort, tacrolimus, tamoxifen, trazodone, verapamil, vinblastine, vincristine and yohimbine and combinations thereof.

“Modifying” the administering of said P-gp modulators shall be understood to mean reducing the dosage or dose frequency of said P-gp modulators.

“Avoiding” completely the administering of said P-gp modulators shall be understood to mean either a) not starting the administration of said P-gp modulators or b) stopping the administration of said P-gp modulator and then not re-starting the administration of said P-gp modulator.

The treatment may also involve a combination of treatments, including, but not limited to a tyrosine kinase inhibitor in combination with other tyrosine kinase inhibitors, chemotherapy, radiation, etc. Reference in this regard may be made to EP 09160202.9, PCT/EP/2010050338 , WO 2008/121467, US 2009-0306101, US 2006-0058311, US 2005-0043233, US 2003-0225079 and US 2009-0318480.

Concomitant use of a P-gp modulator in treatment of a patient with a first medication such as an EGFR inhibitor means that the P-gp modulator is administered to the patient according to a treatment regimen characterized by repeated administration of a dosage unit in defined time intervals, e.g. once, twice or thrice daily, in addition to a parallel but independent treatment regimen characterized by repeated administration of a dosage unit of the first medication. Additionally the concomitant use of a P-gp modulator together with an EGFR inhibitor means that both drugs would be adminsitered in parallel (simultaneously) or at maximum within a time frame of 1 hour between both drug administrations.

EGFR inhibitor naïve cancer patients,are pateints who are EGFR inhibitor naïve are patients who have never been previously exposed to an EGFR inhibitor.

Monitoring tumor progression may be determined by comparison of tumor status between time points after treatment has commenced or by comparison of tumor status between a time point after treatment has commenced to a time point prior to initiation of treatment. Tumor progression may be monitored during treatment visually, for example, by means of radiography, for example, X-ray, CT scan, or other monitoring methods known to the skilled artisan, including palpitation of the cancer or methods to monitor tumor biomarker levels. Tumor progression is assessed by methods known in the art, such as according to the RECIST criteria, as published e.g. in J. Nat. Cancer Inst., Vol 92, No. 3, 2000, pp 205-216, or in J. Clin. Oncol. Vol 24, No. 20, 2006, pp 3245-3251.

The disclosure of WO 02/50043, WO 2004/074263 and WO 2005/037824 includes preparation as well as pharmaceutical formulations of the compounds and is incorporated by reference regarding these aspects. Furthermore, it is known for treatment of tumour diseases that the compounds may be used in monotherapy or in conjunction with other anti-tumour therapeutic agents, for example in combination with topoisomerase inhibitors (e.g. etoposide), mitosis inhibitors (e.g. vinblastine), compounds which interact with nucleic acids (e.g. cisplatin, cyclophosphamide, adriamycin), hormone antagonists (e.g. tamoxifen), inhibitors of metabolic processes (e.g. 5-FU etc.), cytokines (e.g. interferons) or antibodies.

The expression “patient” relates to a human patient suffering from cancer and thus in need of such treatment. Furthermore, the expression “patient” should be understood to include such cancer patients carrying tumors with wild-type EGF receptor as well as pre-selected cancer patients with tumors harboring activating EGFR mutations. These can be located in the tyrosine kinase domain of the EGF receptor such as for instance the L858R or L861 point mutations in the activation loop (exon 21), or in-frame deletion/insertion mutations in the ELREA sequence (exon 19), or substitutions in G719 situated in the nucleotide binding loop (exon 18). Additional activating mutations have been reported in the extracellular domain of the EGF receptor in various indications (e.g. EGFR vIII displaying exon 2-7 deletions). Other mutations such as the T790M point mutation in exon 20 as well as certain exon 20 insertions (e.g. D770_N771insNPG) which confer resistance to particular drugs should also be included, as well as double mutants such as the combined L858R/T790M mutation or the exon-19-del/T790M.

The expression “patient” should be understood to include also such cancer patients carrying tumors with wild-type HER2 receptor as well as pre-selected cancer patients with tumors harboring activating HER2 mutations, e.g. M774_A775insAYVM.

“Customizing” a medicament or a pharmaceutical composition comprising an EGFR inhibitor, such as BIBW2992, as the active product ingredient for avoiding co-administration with P-gp inducers and/or inhibitors shall be understood to mean adding an instruction to the medicament or pharmaceutical composition for avoiding co-administration with P-gp inducers and/or inhibitors. This instruction may be in any form suitable for pharmaceuticals, e.g. in form of a leaflet added to the medicament within secondary packaging or an imprint on the primary or secondary packaging.

The highest dose of an EGFR-inhibitor, preferably of BIBW2992, is 160 mg once daily for 3 days or, alternatively 100 mg once daily for 2 weeks.

The presence of specific gain-of-function mutations within the tyrosine kinase domain of the EGF receptor in a subgroup of NSCLC patients has been associated with increased sensitivity to treatment with gefitinib and erlotinib (Lynch, New England Journal Medicine 350, 2129 (2004); Paez, Science 304, 1497 (2004); Pao, Proceedings of the National Academy of Science of the United States 101, 13306 (2004)). The treatment with EGFR inhibitors may be a first line treatment, or subsequent line treatments such as cancers initially be diagnosed as gefitinib/erlotinib sensitive or predicted to be gefitinib/erlotinib sensitive by means of these methods. In particular, the L858R point mutation (exon 21) as well as deletion/insertion mutations in the ELREA sequence (exon 19) account for the majority of gefitinib responders. A secondary point mutation in exon 20, T790M, is associated with acquired resistance to gefitinib or erlotinib. This mutation is analogous to the T315I mutation identified in CML patients who relapse under imatinib treatment (imatinib resistant patients). Methods for detecting mutations in the tyrosine kinase domain of the EGF receptor are known in the art, several corresponding diagnostic tools are approved by the FDA and commercially available, e.g. an assay for the detection of epidermal growth factor receptor mutations in patients with non-small cell lung cancer (Genzyme Corp.; see also Journal of Clinical Oncology, 2006 ASCO Annual Meeting Proceedings (Post-Meeting Edition). Vol 24, No 18S (June 20 Supplement), 2006: Abstract 10060).

Irreversible inhibitors in contrast to reversible inhibitors (e.g., gefitinib), are able to inhibit proliferation and EGF-induced EGFR phosphorylation in cell lines expressing double mutant EGF receptors (Kwak, Proceedings of the National Academy of Science of the United States 102, 7665 (2005) and Kobayashi, New England Journal Medicine 352, 786 (2005)).

Any aspect of the present invention therefore includes optional pre-selection of cancer patients for an EGFR mutation in the tyrosine kinase domain of the EGF receptor as well as pre-selection of cancer patients for an HER2 mutation. The EGFR mutations preferably relevant in in this context are selected from the group consisting of the L858R and L861 point mutations in the activation loop (exon 21), in-frame deletion/insertion mutations in the ELREA sequence (exon 19), substitutions in G719 situated in the nucleotide binding loop (exon 18), activating mutations in the extracellular domain of the EGF receptor such as EGFR vIII displaying exon 2-7 deletions, the T790M point mutation in exon 20, exon 20 insertions such as D770_N771insNPG, and double mutants such as the combined L858R/T790M mutation and the exon-19-del/T790M. The HER2 mutation preferably relevant in in this context is the M774_A775insAYVM mutation.

Most preferred cancer indications are selected from the group consisting of
Head and neck tumours: SCC, AC, transitional cell cancers, mucoepidermoid cancers, undifferentiated carcinomas;
Colorectal cancers, metastatic or non-metastatic: AC, including hereditary forms of AC, carcinoid, sarcoma;
Pancreatic cancers: AC, including ductal and acinary cancers, papillary, adenosquamous, undifferentiated, tumours of the endocrine pancreas;
Breast cancers, metastatic or non-metastatic: AC, including invasive ductal, lobular and medullary cancers, tubular, mucinous cancers, Paget-carcinoma, inflammatory carcinoma, ductal and lobular carcinoma in situ;
Prostate cancers: AC, small cell, SCC;
Gastric cancers: AC, adenosquamous, anaplastic;
Ovarian cancer;
Non-small cell lung cancers (NSCLC): adenocarcinoma, SCC, spindle cell carcinoma, AC, bronchioalveolar carcinoma, large cell NSCLC, clear cell NSCLC, NSCLC with EGFR mutations
   but especially
Non-small cell lung cancers (NSCLC): SCC, spindle cell carcinoma, AC, bronchioalveolar carcinoma, large cell NSCLC, clear cell NSCLC, especially metastatic, second line patients who have failed at least one prior chemotherapy regimen or 3rd/4th line patients who have received Tarceva or Iressa for at least 12 weeks and then failed.

And more especially cancer is selected from the group consisting of non-small cell lung cancer (NSCLC), head and neck squamous cell carcinoma (HNSCC), malignant glioma, breast cancer, esophageal cancer, gastric cancer, renal cancer, cervical cancer, prostate cancer, ovarian cancer, pancreatic cancer, hepatocellular cancer, and colorectal cancer (CRC), including metastatic forms thereof.

Preferably to be treated by administration of an irreversible EGFR inhibitor selected from the group consisting of:

(a) 4-[(3-chloro-4-fluorophenyl)amino]-6-{[4-(N,N-dimethylamino)-1-oxo-2-buten-1-yl]amino}-7-cyclobutyloxy-quinazoline,

(b) 4-[(3-chloro-4-fluorophenyl)amino]-6-{[4-(N,N-dimethylamino)-1-oxo-2-buten-1-yl]amino}-7-cyclopentyloxy-quinazoline,

(c) 4-[(3-chloro-4-fluorophenyl)amino]-6-{[4-(N,N-dimethylamino)-1-oxo-2-buten-1-yl]amino}-7-((R)-tetrahydrofuran-3-yloxy)-quinazoline,

(d) 4-[(3-chloro-4-fluorophenyl)amino]-6-{[4-(N,N-dimethylamino)-1-oxo-2-buten-1-yl]amino}-7-((S)-tetrahydrofuran-3-yloxy)-quinazoline (BIBW2992),

(e) 4-[(3-chloro-4-fluorophenyl)amino]-6-{[4-(N,N-dimethylamino)-1-oxo-2-buten-1-yl]amino}-7-(tetrahydropyran-4-yloxy)-quinazoline,

(f) 4-[(3-chloro-4-fluorophenyl)amino]-6-{[4-(N,N-dimethylamino)-1-oxo-2-buten-1-yl]amino}-7-[(tetrahydrofuran-2-yl)methoxy]-quinazoline,

(g) 4-[(3-chloro-4-fluorophenyl)amino]-6-{[4-(N,N-dimethylamino)-1-oxo-2-buten-1-yl]amino}-7-[(tetrahydrofuran-3-yl)methoxy]-quinazoline,

(h) 4-[(R)-(1-phenyl-ethyl)amino]-6-{[4-(N,N-dimethylamino)-1-oxo-2-buten-1-yl]amino}-7-cyclopropylmethoxy-quinazoline,

(i) 4-[(3-chloro-4-fluorophenyl)amino]-6-{[4-(morpholin-4-yl)-1-oxo-2-buten-1-yl]amino}-7-[(tetrahydrofuran-2-yl)methoxy]-quinazoline,

(j) 4-[(3-chloro-4-fluorophenyl)amino]-6-{[4-(N,N-dimethylamino)-1-oxo-2-buten-1-yl]amino}-7-[(S)-(tetrahydrofuran-2-yl)methoxy]-quinazoline,

(k) 4-[(3-chloro-4-fluoro-phenyl)amino]-6-{[4-(homomorpholin-4-yl)-1-oxo-2-buten-l-yl]amino}-7-[(S)-(tetrahydrofuran-3-yl)oxy]-quinazoline, and

(r) 4-[(3-chloro-4-fluoro-phenyl)amino]-6-{[4-(dimethylamino)-1 oxo-2-buten-1-yl]amino}-7-cyclopropylmethoxy-quinazoline,

or a pharmaceutically acceptable salt thereof.

Method of Treatment:

A method of treating cancer would include pre-selection of cancer patients for EGFR and/or HER2 mutations and administering a therapeutically effective amount of an

EGFR inhibitor to a pre-selected cancer patient shown to carry an EGFR mutation in the tyrosine kinase domain of the EGF receptor and/or with a tumor harboring an activating HER2 mutation, optionally in combination with chemotherapy, biological therapy including therapeutic antibodies, targeted therapy including mTOR inhibitors, radiotherapy, radio-immunotherapy and/or tumour resection by surgery. The method further comprises modifying or avoiding completely the administering of said P-gp inhibitors or inducers before starting of administering an EGFR inhibitor.

Another method of treating cancer in a cancer patient by administering a therapeutically effective amount of an EGFR inhibitor to said cancer patient, optionally in combination with chemotherapy, biological therapy including therapeutic antibodies, targeted therapy including mTOR inhibitors, radiotherapy, radio-immunotherapy and/or tumour resection by surgery, wherein said cancer patient would was pre-selected based on a) having had at least 12 weeks of treatment with a reversible EGFR inhibitor and b) having failed treatment with said reversible EGFR inhibitor. The method further comprises modifying or avoiding completely the administering of said P-gp inhibitors or inducers before starting of administering an EGFR inhibitor.

The EGFR inhibitor can be optionally in form of its tautomers, racemates, enantiomers, diastereomers and the mixtures thereof and optionally in form of the pharmacologically acceptable acid addition salts, solvates, hydrates, polymorphs or physiologically functional derivatives thereof. The EGFR inhibitor is administered orally, enterically, transdermally, intravenously, peritoneally or by injection, preferably orally. In either case, the P-gp inhibitor/inducer should be avoided and/or modified.

Dosage:

The EGFR inhibitor may be administered to the human patient in a daily dose of 0.01-4 mg/kg of body weight (bw), preferably 0.1-2 mg/kg, particularly preferred in a dose of 0.2-1.3 mg/kg bw. For oral treatment the EGFR inhibitor may be administered daily in a total dose of 10, 20, 30, 40, 50, 60, 70, 100, 200, or 300 mg, optionally divided into multiple doses, e.g. 1 to 3 doses to be administered through the day. Preferably the oral daily dose is administered only once a day. More preferably, the starting dose for BIBW 2992 is 40 mg, preferably in form of a tablet, once daily. For patients, who tolerate a 40 mg starting dose, a dose of 50 mg may be considered. For patients, who already had TKI (tyrosine kinase inhibitor) treatment, the starting dose is 50 mg, preferably in form of a tablet, once daily. Especially for higher doses periods of treatment should alternate with periods of recovery, without administering the active EGFR inhibitor. For instance, treatment could follow a “7 day on—7 day off”, a “14 day on—14 day off”, a “21 day on 7 day off” or a continuous dosing schedule. “On-off” time periods can be chosen shorter, especially if higher doses are administered, or individually adapted to the needs of the patient. Preferable dosage of BIBW 2992 is 20, 30, 40, 50 mg, most preferably 40 mg once daily.

The dosage for intravenous use of a EGFR inhibitor, e.g. of BIBW2992MA2 may be 1-1000 mg, preferably 5-300 mg, particularly preferred 10-100 mg (dosages refer to the base form BIBW2992), either given as a bolus or, especially if higher doses are applied, as a slow intravenous infusion over several hours, e.g. over about 1, 2, 4, 6, 10, 12 or 24 hours.

In one embodiment the invention relates to the method of treatment described above, characterised in that an EGFR inhibitor, or its polymorph, metabolite, hydrate, solvate, an individual optical isomer, mixtures of the individual enantiomers or racemates thereof, or a pharmaceutically acceptable salt thereof, is administered intermittent or in a daily dosage such that the plasma level of the active substance preferably lies between 10 and 5000 nM for at least 12 hours of the dosing interval.

However, it may optionally be necessary to deviate from the amounts specified, depending on the body weight or method of administration, the individual response to the medication, the nature of the formulation used and the time or interval over which it is administered. Thus, in some cases, it may be sufficient to use less than the minimum quantity specified above, while in other cases the upper limit specified will have to be exceeded. When large amounts are administered it may be advisable to spread them over the day in a number of single doses.

Moreover, the recommended intake for an EGFR inhibitor, preferable BIBW 2992, is without food and at least one hour before a meal or at least 3 hours after a meal.

An EGFR inhibitor, its tautomers, the racemates, the enantiomers, the diastereomers and the mixtures thereof, and optionally the pharmacologically acceptable acid addition salts, solvates, hydrates, polymorphs, physiologically functional derivatives or prodrugs thereof, may be used in monotherapy or combined with other active substances according to the invention, optionally also in conjunction with other pharmacologically active substances.

Pharmaceutical Formulations:

Suitable pharmaceutical preparations for the use in accordance with the invention include, for example, tablets, capsules, suppositories, solutions, and particularly solutions for injection (s.c., i.v., i.m.) and infusion, syrups, emulsions or dispersible powders. The amount of pharmaceutically active compound in each case should be in the range from 0.1-90 wt. %, preferably 0.5-50 wt. % of the total composition, i.e. in amounts which are sufficient to achieve the dosage range given below. The doses specified may, if necessary, be given several times a day.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharin, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p hydroxybenzoates.

Solutions for injection and infusion are prepared in the usual way, e.g. with the addition of preservatives such as p hydroxybenzoates, or stabilisers such as alkali metal salts of ethylenediamine tetraacetic acid, optionally using emulsifiers and/or dispersants, while if water is used as the diluent organic solvents may optionally be used as solubilisers or auxiliary solvents, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Suitable excipients may be, for example, water, pharmaceutically acceptable organic solvents, such as paraffins (e.g. petroleum fractions), oils of vegetable origin (e.g. groundnut or sesame oil), mono- or polyfunctional alcohols (e.g. ethanol or glycerol), carriers such as e.g. natural mineral powders (e.g. kaolin, clays, talc, chalk), synthetic mineral powders (e.g. highly dispersed silica and silicates), sugar (e.g. glucose, lactose and dextrose), emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered in the usual way, preferably by oral or transdermal route, particularly preferably by oral route. When administered orally the tablets may, of course, contain additives, such as e.g. sodium citrate, calcium carbonate and dicalcium phosphate together with various additives, such as starch, preferably potato starch, gelatine and the like, in addition to the abovementioned carriers. Lubricants such as magnesium stearate, sodium laurylsulphate and talc may also be used to form tablets. In the case of aqueous suspensions the active substances may be combined with various flavour enhancers or colourings in addition to the abovementioned excipients. For parenteral use, solutions of the active substances may be prepared using suitable liquid carrier materials.

Preferred formulations and drug forms for BIBW 2992 are disclosed in WO2005/037824 WO2009147238 and WO2011003853.

### EXAMPLES

The following examples are not intended, nor are they to be construed, as limiting the invention.

### Example 1

### Methods of Making EGFR Inhibitors

Methods of making any of the above mentioned EGFR inhibitors are known in the art. Specific working examples of BIBW 2992 can be found in U.S. Pat. No. 7,019,012, WO2005/037824 and WO2007/085638.

### Example 2

### Preclinical Data Indicated that BIBW2992 is a Substrate of P-Glycoprotein (P-gp)

Passive Permeability through CaCo-2 Cell Layers and P-gp Transport Profiling:

Experiments were performed to assess its passive permeability and potential transport by P-glycoprotein (a.k.a. MDR1, ABCB1) and the potential inhibition of P-gp by BIBW 2992.

B1BW 2992 exhibited high passive permeability and was a substrate of P-gp (estimated Km 10-30 uM) as well as an inhibitor of P-gp with an estimated Kᵢ of 3.4 μM (mean of two independent experiments).

### Example 3

### Clinical Data Indicated that BIBW2992 is a Substrate of P-Glycoprotein (P-gp)

Since BIBW 2992 was found to be a P-gp substrate in vitro (see above), investigators performed a phase I trial in healthy volunteers to assess the effects of the potent P-gp inhibitor ritonavir on the pharmacokinetics (PK) of BIBW 2992 (a P-gp substrate). In this open-label, randomised, two-way crossover study the relative exposure after a single oral dose of B1BW 2992 (20 mg), co-administered with multiple oral doses of ritonavir (200 mg bid for 3 days), was compared to the exposure after a single oral dose of BIBW 2992 (20 mg) alone in healthy male volunteers. The study was designed to determine the maximum effect of P-gp inhibition on the PK of BIBW 2992.

When BIBW 2992 20 mg once daily was given in combination with ritonavir 200 mg twice daily, AUC_{0-∞} of BIBW 2992 increased by 47.6% (90% CI 133.7%, 162.9%),AUC_{0-tz} increased by 49.0% (90% CI 134.5%, 165.1%), and Cₘₐₓ increased by38.5% (90% CI 120.6%, 158.9%) compared with BIBW 2992 given alone. This was a surprising result. Due to this relatively high concentration in the micromolar range in correlation to the average maximum B1BW 2992 plasma concentrations at steady state (I/Kᵢ<0.1), drug-drug interactions based on inhibition of P-gp by BIBW 2992 had been considered as less likely to occur. Median tₘₐₓ of BIBW 2992 was 4.00 hours with and without ritonavir. The distribution and elimination phases of BIBW 2992 appeared to be unaffected by ritonavir cotreatment. Also, the terminal half-life of BIBW 2992 was unchanged.

Since previous studies revealed that CYP3A4 enzyme-catalysed metabolic reactions play a subordinate role for the metabolism of BIBW 2992 in vivo and CYP3A4-dependent N-demethylation of BIBW 2992 was too low to be quantitatively detected in healthy volunteers, the increase in BIBW 2992 exposure in the presence of ritonavir is most likely attributed to the inhibition of P-gp-mediated transport processes during the absorption phase of BIBW 2992.

Because of these results, the following measures were implemented for clinical trials of BIBW 2992 as a protocol amendment which indicted that:

The use of potent P-gp inhibitors (including preferably Cyclosporin, Erythromycin, Ketoconazole, Itraconazole, Quinidine, Phenobarbital salt with Quinidine, Ritonavir, Valspodar, Verapamil) and potent P-gp inducers (including preferably St John's wort, Rifampicin) has to be avoided during treatment with BIBW 2992.

### Example 4

### Administration of Both BIBW 2992 and a P-gp Inducer

If a potent P-gp inducer is given with BIBW 2992, then the opposite effect is most likely to occur than for a P-gp inhibitor, i.e., there is a lowering of the BIBW 2992 drug exposure.

### Example 5

### Treatment of Lung Cancer Patients Using BIBW 2992 Following Failure with a Reversible EGFR Inhibitor

Patients with adenocarcinoma of the lung who had previously failed a reversible EGFR inhibitor (e.g., gefitinib or erlotinib) and who had had at least 12 weeks of therapy with said reversible inhibitor are identified as being candidates for treatment with BIBW 2992. However, prior to treatment with BIBW 2992, certain patients are found to be taking a potent P-gp inhibitor or P-gp inducer. Prior to starting BIBW 2992 treatment, treatment with the potent P-gp inhibitor or P-gp inducers is stopped. Following this cessation of P-gp inhibitor or P-gp inducer therapy, BIBW 2992 treatment is then started at a dose of 50 mg daily.

### Example 6

### BIBW 2992 Treatment of Lung Cancer Patients Having Tumors with EGFR Mutations

Patients with adenocarcinoma of the lung who have EGFR mutations (including, but not limited to deletion-19 mutations, other mutations in exon 19, L858R mutations, other mutations in exon 21, mutations in exon 18 and mutations in exon 20) are identified as being candidates for treatment with BIBW 2992. However, prior to treatment with BIBW 2992, certain patients are found to be taking a potent P-gp inhibitor or P-gp inducer. Prior to starting BIBW 2992 treatment, treatment with the potent P-gp inhibitor or P-gp inducers is stopped. Following this cessation of P-gp inhibitor or P-gp inducer therapy, BIBW 2992 treatment is then started at a dose of 50 mg daily.

### Example 7

### BIBW 2992 Treatment of Head-and-Neck Cancer Patients

Prior to treatment of head-and-neck cancer patients with BIBW 2992, certain patients are found to be taking a potent P-gp inhibitor or P-gp inducer. Prior to starting BIBW 2992 treatment, treatment with the potent P-gp inhibitor or P-gp inducers is stopped. Following this cessation of P-gp inhibitor or P-gp inducer therapy, BIBW 2992 treatment is then started at a dose of 50 mg daily.

### Example 8

### BIBW 2992 Treatment of Breast Cancer Patients Having Tumors with HER2 Overexpression or HER2 Gene Amplification

Patients with breast cancer who have HER2 overexpression or HER2 gene amplification are identified by methods known in the art such as immunohistochemistry or FISH are identified as being candidates for treatment with BIBW 2992. However, prior to treatment with BIBW 2992, certain patients are found to be taking a potent P-gp inhibitor or P-gp inducer. Prior to starting BIBW 2992 treatment, treatment with the potent P-gp inhibitor or P-gp inducers is stopped. Following this cessation of P-gp inhibitor or P-gp inducer therapy, BIBW 2992 treatment is then started at a dose of 40 mg daily.

All patent documents and non-patent literature are incorporated herein by reference in their entirety.

1. A method of treatment with an EGRF inhibitor, the method comprising
(a) identifying a patient in need of treatment with an EGRF inhibitor;
(b) determining that the patient is receiving therapy with a P-gp modulator;
(c) causing the patient's therapy with the P-gp modulator to cease for the duration of treatment with the EGRF inhibitor; and
(d) administering the EGRF inhibitor to the patient.

2. A method of treatment with an EGRF inhibitor, the method comprising
(a) identifying a patient in need of treatment with an EGRF inhibitor;
(b) determining that the patient is receiving therapy with a P-gp modulator;
(c) modifying the administering of said P-gp modulator before starting of administering the EGFR inhibitor; and
(d) administering the EGRF inhibitor to the patient.

3. The method of claim 1, wherein the P-gp modulator is a P-gp inhibitor.

4. The method of claim 1, wherein the P-gp modulator is a P-gp inducer.

5. The method of claim 1, wherein the EGFR inhibitor is a reversible EGFR inhibitor.

6. The method of claim 2 wherein the EGFR inhibitor is an irreversible EGFR inhibitor.

7. The method of claim 2 wherein the EGFR inhibitor is BIBW2992 or a pharmaceutically acceptable salt thereof.

8. A method of treatment with BIBW 2992, the method comprising
(a) administering BIBW 2992 to a patient who is receiving therapy with a P-gp modulator;
(b) detecting symptoms of BIBW 2992 adverse events in the patient; and
(c) reducing or ceasing therapy with the P-gp modulator, while continuing therapy with BIBW 2992.

9. A method of treatment with BIBW 2992, the method comprising
(a) identifying a patient in need of treatment with BIBW 2992;
(b) providing the patient and/or the patient's caregiver with a list of drugs that are P-gp modulators;
(c) advising the patient and/or the patient's caregiver not to treat the patient with any of the listed drugs for the duration of treatment with BIBW 2992; and
(d) administering BIBW 2992 to the patient.

10-22. (canceled)

23. An in vitro method for diagnosis of a patient who is eligible for treatment with an EGFR inhibitor, wherein the method comprises the following steps:
determining in a sample obtained from the patient whether the patient has a tumor expressing wild type or mutated forms of EGFR, and determining from data collected regarding said patient whether the patient is not under a P-gp modulator medication;
and wherein the patient is eligible for EGFR inhibitor treatment only if both above determinations can be answered affirmative.

24. A method for monitoring cancer during an EGFR inhibitor treatment, wherein said method comprises the following steps:
providing a sample obtained from the patient to monitoring the progression or regression of disease during the EGFR inhibitor treatment, and
discontinuing a P-gp modulator treatment or modifying the daily dosage thereof or increasing the daily dosage of the EGFR inhibitor.

25. A method of treatment with an EGFR inhibitor, the method comprising
(a) identifying a patient in need of treatment with an EGFR inhibitor;
(b) determining that the patient is receiving therapy with a P-gp modulator;
(c) administering simultaneously of a P-gp inhibitor/inducer together with a EGFR inhibitor; and
(d) modifying the simultaneous administration of a P-gp inhibitor/inducer together with a EGFR inhibitor or the dose of the EGFR inhibitor.

26. The method of claim 25 wherein the modifying the simultaneous administration of a P-gp inhibitor/inducer together with a EGFR inhibitor is done by reducing the dosage or dose frequency of said P-gp inhibitor/inducer.

27. A method of treatment with an EGRF inhibitor, the method comprising
(a) identifying a patient in need of treatment with an EGRF inhibitor, preferably BIBW 2992;
(b) determining that the patient is receiving therapy with a P-gp modulator;
(c) causing the patient's therapy with the P-gp modulator to cease prior to treatment with the EGRF inhibitor;
(d) administering the EGRF inhibitor to the patient;
(e) resuming therapy with a P-gp modulator not earlier than 6 hours after the EGRF inhibitor administration.

28. The method of claim 1 wherein the EGFR inhibitor is BIBW2992 or a pharmaceutically acceptable salt thereof.
